(19) United States
(12) Patent Application Publication
(10) Pub. No.: US 2003/0139371 A1
(43) Pub. Date: Jul. 24, 2003

(54) Drug for combating sexual dysfunctions

(76) Inventors: Philippe Gorny, Paris (FR); Catherine Pons-Himbert, Gonesse (FR); Olivier Stucker, Paris (FR)

Correspondence Address: IP DEPARTMENT OF PIPER RUDNICK LLP, 3400 TWO LOGAN SQUARE, 18TH AND ARCH STREETS, PHILADELPHIA, PA, 19103, US

(21) Appl. No.: 10/360,354

(22) Filed: Feb. 7, 2003

Related U.S. Application Data
(63) Continuation of application No. PCT/FR01/02579, filed on Aug. 8, 2001.

(30) Foreign Application Priority Data
Aug. 8, 2000 (FR) FR 00/10435

(51) Int. Cl. A61K 31/7076; A61K 31/522

(52) U.S. Cl. 514/46; 514/47; 514/263.32; 514/161; 514/263.31

(57) ABSTRACT

A composition which includes a purine, an NSAID, and a pharmaceutical excipient and/or vehicle. A method which treats male or female sexual dysfunction and includes administering a therapeutically effective amount of the composition to a person. A method which prevents male or female sexual dysfunction and includes administering a therapeutically effective amount of the composition to a person, and a method for increasing sexual desire and/or promoting sexual activity and/or increasing sexual capacities and/or promoting sexual activity and/or improving the intensity of sexual pleasure and/or promoting the attainment of satisfying sexual relations in persons not suffering from sexual dysfunctions and includes administering to a person a composition including a purine and an NSAID.

### RELATED APPLICATION

This application is a continuation of International Patent Application No. PCT/FR01/02579, filed Aug. 8, 2001, which claims priority of French Patent Application No. 00/10435, filed Aug. 8, 2000.

### FIELD OF THE INVENTION

This invention pertains to a drug intended especially to prevent or treat sexual dysfunctions in men or women. The invention pertains in particular to the production of a drug capable of combating disorders in the physiological and/or anatomical response to sexual stimulation in humans. Such a drug contains in combination a purine and a nonsteroidal anti-inflammatory agent.

### BACKGROUND

The erectile tissue of the penis, called the corpus cavernosum, is a spongy tissue capable of becoming filled with blood. When at rest, the arteries of the penis are dependent on the adrenergic tonus which maintains them in a hypertonic state such that no noteworthy blood flow fills the corpus cavernosum. In the case of appropriate stimulation, the erector nerves inhibit the adrenergic tonus, liberate certain mediators promoting the dilation of the arteries of the penis, which leads to an accumulation of blood in the corpus cavernosum. The penis becomes enlarged while the augmentation of the internal pressure causes it to become rigid. As it enlarges, the penis presses the cavernous veins against the envelope of the erectile body, thereby preventing evacuation of the blood that it contains and ensuring maintenance of its rigidity. After ejaculation, noradrenaline is again liberated locally, thereby causing a reduction in arterial blood supply such that the pressure in the corpus cavernosum diminishes and the blood accumulated in the corpus can be evacuated via the veins which are no longer compressed, which leads to the loss of rigidity and a return to the resting state.

In women, sexual excitation is manifested notably by the vasodilation of the blood vessels irrigating the genital organs. This vasodilation leads in particular to a swelling and erectile response of the clitoris, as well as vasocongestion of the vaginal wall with exudation of vaginal fluids.

It is known that a rather considerable proportion of men (between 10 and 50%, depending on the populations studied and the age groups) suffer from permanent or temporary erectile dysfunction. These disorders can be of organic origin, in which case specific treatments adapted to each situation are required. But it has also been seen that a majority of erectile dysfunctions are not organic, but often of psychogenic origin; see, e.g., Feldman H. A. et al., J. Urol. 151; 54-61 (1994).

In women as well, the physiological response to sexual stimulation and its anatomical manifestation can deteriorate temporarily, and sometimes permanently, even without detectable organic cause. The most frequently observed disorders include the absence of sexual desire even after stimulation, difficulty in achieving orgasm, a low level of sexual pleasure and a decrease in natural vaginal lubrication or even an absence thereof. The consequence of these disorders is often a lack of interest in sexual activity. These disorders in the physiological and/or anatomical response to sexual stimulation are sometimes hereinafter referred to as “female sexual dysfunctions”. According to certain estimates, the frequency of temporary or chronic sexual dysfunctions in women is equivalent to that of erectile dysfunctions in men; see, e.g., Laumann E. O., JAMA 281; 537-544 (1999).

It is, therefore, desirable to have available treatments making it possible to reduce the severity and/or duration of these disorders, or to prevent their occurrence, so as to restore the capacity of attaining satisfying sexual relations in male or female subjects who have such disorders or who fear their occurrence.

The physiology of the erection, and more generally the phenomenon of turgidity of the erectile bodies (penis, clitoris), is a complex phenomenon combining neuronal and vascular mediators. The erection is maintained by the relaxation of the afferent arteries to the corpus cavernosum and the smooth muscles of this corpus cavernosum.

Among the molecules inducing relaxation is found nitrogen monoxide (NO) liberated by the vascular endothelium and by NANC (nonadrenergic noncholinergic) nerve fibers.

It has been established that nitrogen monoxide stimulates the synthesis of cyclic guanosine monophosphate (cGMP) which is the effective agent of muscle relaxation of the arteries. It is also known that nitrogen monoxide is the principal physiologic neurotransmitter brought into play by the nonadrenergic and noncholinergic peripheral neurons enervating the corpus cavernosum and its arteries, and that the liberation of nitrogen monoxide at the level of the effector synapse is an important factor in the induction of the erection; see especially BURNETT et al., Science 257: 401-403 (1992), and FAJFER et al., New Engl. J. Med. 326: 90-94 (1992).

It is known that prostaglandins have a regulatory effect on the tonus of the cavernous muscles, either by inducing vasodilation (prostaglandin I2, prostaglandin E2) or by inducing vasoconstriction (prostaglandin F2 alpha).

Moreover, the purines also play an important role in the vascular control of the erection. They intervene especially via the intermediary of specific receptors. It has been demonstrated in the rabbit that purines are capable of inducing relaxation of the corpus cavernosum; see WU H-Y et al., Int. J. Impotence Res. 5, 161-167 (1993). It has also been demonstrated that the intravenous injection of adenosine triphosphate induces an erection in dogs; see TAKAHASHI Y et al., Int. J. Impotence Res. 4, 27-34 (1992).

### SUMMARY OF THE INVENTION

This invention relates to a composition including a purine, an NSAID, and a pharmaceutical excipient and/or vehicle.

This invention also relates to a method of treating male or female sexual dysfunction including administering a therapeutically effective amount of the composition to a person.

This invention further relates to a method preventing male or female sexual dysfunction including administering a therapeutically effective amount of the composition to a person.

This invention still further relates to a method for increasing sexual desire and/or promoting sexual activity and/or increasing sexual capacities and/or promoting sexual activity and/or improving the intensity of sexual pleasure and/or promoting the attainment of satisfying sexual relations in persons not suffering from sexual dysfunctions including administering to a person a composition including a purine and an NSAID.

### DETAILED DESCRIPTION

We attempted to discover whether other mediators had a regulatory effect on the action of purines.

The in vitro model we used was that of the isolated rabbit corpus cavernosum in organ chambers. A good similarity of response has been demonstrated with the human corpus cavernosum. In fact, the best correlations with the results found in humans have been found in the rabbit; see, e.g., Bush P. A., Aronson W. J., Buga G. M., Rajfer J., Ignarro L. J., J. Urol. 147(6); 1650-1655 (1992); Knispel H. H., Goessel C., Bechman R., Urol. Res. 20(4); 253-257 (1992); Holmquist F., Hedlund H., Andersson K. E., J. Physiol. (London) 449; 295-311 (1992); and Cellek S., Moncada S., Proc. Natl. Acad. Sci. USA 94(15); 8226-8231 (1997).

The relaxant action of purines was studied in this model either with purines alone or with an inhibitor of NO synthesis (N-omega-nitro-L-arginine, or L-NNA) so as to investigate whether the regulation by purines of the production of NO is a component of the relaxant effect of purines. These studies showed that the presence of an inhibitor of the synthesis of NO diminished slightly, by only about 15%, the effects observed with purine alone.

In a similar manner, we investigated whether the presence of an inhibitor of the synthesis of prostaglandins (cyclooxygenase inhibitor) modified the effects of purine. If the action of purine could be explained in part by an indirect effect via prostaglandins, we would expect to see a decrease in the effect of purine in the presence of the cyclooxygenase inhibitor.

However, we found that to the contrary and in a surprising manner, the cyclooxygenase inhibitors strongly potentiated the relaxant effect of purine and diminished the vasoconstrictive effect of the catecholamines. This discovery was especially surprising since the cyclooxygenase inhibitor by itself had no relaxant effect on the smooth muscle at rest.

The nonsteroidal anti-inflammatory drugs (NSAIDs) possess in common diverse properties, notably an inhibitory effect on cyclooxygenase. It was with aspirin, a very well known nonsteroidal anti-inflammatory drug, that the study referenced above was performed. The results were confirmed with other NSAIDs, notably salicylic acid, mefenamic acid and indomethacin.

Thus, the combination of a purine activity and a nonsteroidal anti-inflammatory agent activity provides favorable results in the prevention and treatment of disturbances in the physiological and anatomical response to sexual stimulation in humans (men and women) and, thus, makes it possible to combat these disorders by means of a synergistic effect.

Thus,the invention relates to a drug combining purine activity and NSAID activity, and also comprising a pharmaceutical excipient or vehicle. The drug of the invention generally contains at least one purine and at least one NSAID.

“Purine” is understood to mean especially puric bases, notably adenine, the purine-based nucleosides and notably adenosine as well as corresponding phosphates, including but not limited to AMP, ADP and ATP or guanine, guanosine, GMP, GDP, GTP and their derivatives, notably their pharmaceutically acceptable salts (for example, adenine or adenosine hydrochloride, or sodium salts of adenosine-phosphates). “Purine” is more generally also understood to mean any substance capable of acting on the purinic receptors (notably P1 receptors sensitive to AMP and adenosine, and P2 receptors sensitive to ADP and ATP). Such substances are known or can be found by known methods. “Purine activity” is an activity obtained by the presence of a purine such as defined above.

Nonsteroidal anti-inflammatory drugs or NSAIDs constitute a known class of anti-inflammatory agents; see, e.g., THE MERCK INDEX, 12^{th} edition, the content of which regarding NSAIDs (including the data and references) is incorporated herein by reference. The NSAIDs have many properties in common: a cyclooxygenase inhibition activity which gives them the capacity to inhibit the synthesis of prostaglandins. The NSAIDs have other properties in common: the decoupling of oxidative phosphorylation, modifications of the intracellular movements of calcium ions, activation of the synthesis of inducible NO synthase, action on the kappa nuclear factors and the like. It is possible that one or more of these properties is responsible for the potentiating effect of the NSAIDs on purines, but it is also possible that other known or unknown properties are involved. “NSAID activity” is an activity obtained by the presence of a product having at least one of the common properties of the NSAIDs.

Among the NSAIDs that are preferred, we can cite especially: salicylic derivatives such as acetylsalicylic acid (aspirin), methyl salicylate, salicylic acid, 2-(2-nitroxy)-butyl 2-acetoxybenzoate and 2(2-nitroxymethyl) phenyl 2-acetoxybenzoate; pyrazole derivatives such as phenylbutazone, tolmetin, antipyrine, noramidopyrine, dipyrone, oxyphenbutazone, azapropazone, bumadizone, clofezone, kebuzone, mofebutazone, proxifezone, pyrazinophenazone, suxibuzone; anthranilic acid derivatives (also called fenamates) such as mefenamic acid, flufenamic acid, niflumic acid, tolfenamic acid, meclofenamic acid, etofenamic acid; propionic acid derivatives such as: ibuprofen, ketoprofen, maproxen, fenoprofen, flurbiprofen, tiaprofenic acid, naproxen; phenothiazine derivatives such as methiazinic acid or protizinic acid; other organic acid derivatives such as bucloxic acid, diclofenac or piroxicam; indole derivatives such as indomethacin or sulindac; NSAIDs selectively or preferentially inhibiting cyclooxygenase-2 (or Cox-2) such as rofecoxib, celecoxib or nabumetone; NSAID nitrogen monoxide donor derivatives, notably the nitric esters and the nitro or nitroso derivates described in the patents and patent applications EP 0 670 825, U.S. Pat. No. 5,700,947, WO 95/30641, U.S. Pat. Nos. 5,703,073, 6,043,232 and 6,043,22, the contents of which are incorporated herein by reference.

It is, of course, possible to use any other NSAID (having the capacity of potentiating the action of purines) such as the NSAIDs described in THE MERCK INDEX, 12^{th} edition.

Generally speaking herein, “derivatives” refer to all products obtained by the modification of a chemical functional group or an atom or a group of atoms of an active product, and which have a physiological activity of the same type as the active product. As examples, the derivatives of active products having acid functional groups can be notably the salts (for example, sodium salts or salts of other alkaline metals, or salts formed with amines, e.g., piperazine salts or lysine salts), or the esters formed by said acids with alcohols, or the amides formed by these acids with amines; the derivatives of active products having amine functional groups are notably the amides and the addition salts formed by these amines with the acids; the derivatives of active products having alcohol functional groups are notably the esters formed by the alcohols with the acids.

In order to study the effects of agents intended to combat disorders in the physiological and anatomical response to sexual stimulation in men and women, it is possible to use known methods described in the literature, e.g., those described by Boolell M. et al., Intern. Journal of Impotence Research 8; 47-52 (1996) and by Goldstein I., New England J. of Medicine 338; 20, 1397-1404 (1998), or by means of the techniques and organ chamber mentioned below.

The drug of the invention is used in a manner to administer to the treated person therapeutically effective doses which can be determined by simple routine experiments using, e.g., the tests which have already been mentioned. It should also be noted that the active doses of many purines are already known. It is moreover easy to determine the effective doses by means of such tests. The NSAID doses can be easily determined by routine tests, including the tests such as described below employing the isolated rabbit organ.

Thus, the invention uses purine activity and NSAID activity in the preparation of a drug intended to combat male or female sexual dysfunctions, including disorders in the physiological and/or anatomical response to sexual stimulation and, in particular, to prevent or treat nonorganic erectile dysfunctions. This drug can be administered on a curative or preventive basis to subjects who need them, i.e., persons having experienced or who fear the occurrence of such disorders.

The active ingredients of a drug obtained in accordance with the invention can be separated, each in a suitable pharmacological form, or packaged together in the same package. To facilitate simultaneous administration of the active ingredients it is generally preferred to prepare the drug in a single pharmaceutical form containing both active ingredients as well as possibly a suitable pharmaceutical excipient.

A product that has both purine activity and NSAID activity should be considered to itself constitute a combination having the two types of activity, and as such can be used in accordance with the invention as a single active ingredient. For example, a purine and an NSAID can be combined by establishing a chemical bond between the two molecules. It is possible notably to amidify an amine function of the purine base with an acid group present in an NSAID with a carboxylic functional group such as, e.g., acetylsalicylic acid or mefenamic acid. One thereby obtains an amidification product that possesses both purine activity and NSAID activity.

It is, thus, possible to replace the combination of a purine and an NSAID by a single product in which a purine, or a purine analogue, is bound by covalence to an NSAID, possibly by the intermediary of at least one spacer arm.

These products are notably those according to formula I

(A—)ₘ(X)ₚ(—B)ₙ (I)

wherein A is the residue of an NSAID molecule, B is the residue of a purine and X represents either a covalent bond between A and B, or a spacer arm linking at least one A residue with at least one B residue, m is a whole number ranging from 1 to 3, n is a whole number ranging from 1 to 3, and p represents zero or a whole number equal at most to the larger of the numbers m and n. It is possible, depending on the case, to either graft one or more A and/or B residues on a single spacer arm, or graft one or more A—X— groups on a B residue (and then m=p and n=1), or graft one or more —X—B groups on an A residue (and then n=p and m=1). When p=zero, either one or more A residues are linked to a B residue (and n=1), or one or more B residues are linked to an A residue (and m=1).

The products of formula I can be used in the form of salts, particularly in the form of alkaline metal salts such as sodium or potassium salts. These salts are, e.g., those of the phosphate groups if they are present, the phenolic groups (the case of salicylic acid) and the like. It is also possible to use the products of formula I, where appropriate, in the form of addition salts (e.g., in hydrochloride form) when these products contain an amine group.

The bonds between the spacer arm and the A and B residues are covalent bonds. The chemical groups creating the link between A and B (when p=zero), or between A and X or between X and B (when p is other than zero), are, e.g., carboxylic ester, carboxylic amide, thiocarboxylic ester or thiocarboxylic amide groups.

In formula I, A can represent notably the acyl residue of an NSAID possessing a carboxylic group (the NSAID would, thus, have the formula A—OH) and B can represent the residue of a purine base nucleoside or nucleotide bound to X, or bound to A (in the case of absence of spacer arm), by the intermediary of the nitrogen of a primary amine of the purine base and/or by the intermediary of the oxygen of a hydroxyl group of the purine base nucleoside or nucleotide. For example, one or more A or A—X— groups can be linked to B by the intermediary of the oxygen of the primary alcohol of said nucleoside and/or by the intermediary of the oxygen of at least one secondary alcohol of said nucleotide. In these cases the purine from which B is derived obviously has as its formula BH.

In formula I, the nucleoside or nucleotide is notably a ribonucleoside or ribonucleotide. The purine can be selected from among adenosine, guanosine and inosine, as well as the corresponding 5′-monophosphates, -diphosphates and -triphosphates.

The spacer arms can be notably bivalent residues of bifunctional aliphatic compounds (i.e., compounds having at each of their ends reactive functional groups enabling formation of covalent bonds with A and with B). These compounds can be, e.g., compounds that possess both an amino group and a carboxylic (or thiocarboxylic) group, or rather compounds that possess both an amino group and a hydroxyl group.

In formula I, the group X (leaving aside these end functional groups) represents notably a divalent aliphatic group possibly interrupted by one or more —O— or —S— heteroatoms or by one or more —NH— or —CO—NH— heteroatomic groups.

The spacer agents, i.e., the compounds capable of yielding, after reaction with the purine and NSAID, products of formula I in which A and B are linked by spacer arms, are, e.g., alpha-, beta- or gamma-amino alkanecarboxylic acids, in particular, the natural alpha-amino acids such as glycine, alanine, valine or leucine, or peptides, notably dipeptides or tripeptides.

The spacer agents can also be hydroxycarboxylic acids such as lactic acids, glycolic acids, aldonic acids (gluconic, mannonic, galactonic, ribonic, arabinonic, xylonic and erythronic acid) and the corresponding lactones or dilactones (e.g., lactide, glycolide, delta-glucolonactone, delta-valeronactone), or aldaric acids.

The functional groups possibly have on the spacer arm and not involved in the bond with an A or B element can be used for grafting other A and/or B residues to obtain compounds of formula I for which m and/or n is greater than 1. This is the case, for example, with the hydroxyl groups of hydroxy acids, the second carboxylic group of amino diacid carboxylic acids, the second amino group of diamininated amino acids, the hydroxyl group of hydroxylated amino acids.

The classic methods of organic synthesis are used to prepare the compounds of formula I. For example, to prepare amides or esters, one can react a carboxylic compound (NSAID or spacer agent) in the form of a carboxylic (or thiocarboxylic) acid halide or in the form of a mixed anhydride or in the form of an activated ester, e.g., an ester of p-nitrophenyl. The acid can also be activated by means of a coupling agent such as dicyclohexylcarbodiimide.

Since the compounds of formula I comprise residues of nucleosides or nucleotides, they can be prepared using, in particular, the methods known in nucleic acid chemistry, described for example in the publication by Kochetkoc and Budovskii, Organic Chemistry of Nucleic Acids, Plenum Press, 1971 (2 volumes), the contents of which are incorporated herein by reference.

It is, of course, clear that when the compounds from which derive A, B or X of formula I comprise multiple functional groups capable of reacting that it is appropriate to operate either using the reagents in stoichiometric proportions (according to the number of precursor products of A and/or B that it is desired to react), or by temporarily protecting the reactive functional groups that one does not want to react. For this, use is made of temporary protection methods for the reactive functional groups. These temporary protection methods are well known, notably those that were developed in research focused on peptide synthesis. For example, —NH₂ groups can be protected by carbobenzoxy, phthaloyl, t-butoxycarbonyl, trifluoroacetyl or toluenesulfonyl groups; carboxylic groups can be protected in the form of benzyl esters, tetrahydropyranyl esters or t-butyl esters; alcohols can be protected in the form of esters (e.g., acetates), in the form of tetrahydropyranyl ethers, benzyl ethers or trityl ethers, or in the form of acetals (including in the form of acetonides in the case of vicinal glycols). The protection and possible deprotection reactions of various chemical groups are known and described, e.g., in the publication Advances in Organic Chemistry, Methods and Results, Vol. 3, Interscience Publishers (1963), pages 159 and following and pages 191 and following, as well as in the publication by T. W. Green, Protective Groups in Organic Synthesis, Wiley-Interscience Publication (1991). The contents of these publications are incorporated herein by reference.

The phosphatation or dephosphatation reactions of the primary alcohol of nucleotides or nucleosides can be implemented using natural enzymes (e.g., phosphatases, phosphokinases).

Among the products of formula I can be cited, in particular, those according to formula Ia:

A—B (Ia),

in which A and B are defined as above. A represents notably the acyl residue of an NSAID possessing a carboxylic group, the bond with B being made, e.g. by formation of an amide or an ester with an amine or alcohol functional group, respectively, of the purine of formula BH.

Among the products of formula I or Ia, we can cite notably the amides and esters formed with the acyl A residues of salicylic acid, acetylsalicylic acid, diclofenac, ibuprofen, naproxen or sulindac, and with the B residues derived from adenosine or AMP.

It is understood that it is of particular value to select among the products of formula I those that have a potentiating synergic effect in relation to their purine and NSAID constituents. Such products can be selected by simple routine experiments.

It should also be noted that the products of formula I or Ia in general have improved gastric tolerance compared to the NSAIDs from which they are derived.

Among the products of formula I, we can cite notably the product of amidification of AMP by salicylic acid or acetylsalicylic acid, and the product of amidification of adenosine by salicylic acid.

The drug obtained according to the invention can be administered via the oral, sublingual, nasal, pulmonary, vaginal, rectal or transdermal route, or by intracavernous injection.

For this purpose, the drug can be in any form enabling administration via the oral route (in particular in the form of capsules, drinkable solutions or emulsions, powders, gels, granules, tablets or compressed tablets), via the nasal route (e.g., solutions to be administered in the form of drops or sprays), via the pulmonary route (solutions in pressurized aerosol containers), via the rectal route (suppositories), via the cutaneous route (e.g., creams, unguents or transdermal devices, referred to as patches), or via the transmucosal route such as, e.g., via the sublingual route (solutions in pressurized containers or tablets for buccal dissolution) or via the vaginal route (notably gynecological creams or pessaries), or via the intracavernous route (injectable suspensions or solutions).

These pharmaceutical forms are prepared in the conventional manner and can contain appropriate conventional excipients and vehicles.

The drug of the invention can produce favorable results in men suffering from temporary erectile dysfunctions as well as in subjects with chronic erectile dysfunctions. In women, improvements can be observed notably for at least one of the following disorders: decreased or loss of sexual desire, absence of orgasm or difficulty in obtaining an orgasm, vaginal dryness, decrease in the intensity of sexual pleasure and the like.

The drug obtained according to the invention can be used either over long periods of time in the case of chronic erectile dysfunctions (e.g., curative treatments lasting several weeks, several times per year), or in episodic curative treatments in the case of temporary and/or recent erectile dysfunctions, or on an ad hoc basis as needed.

Such a drug can be prepared, e.g., in a pharmaceutical form allowing administration of about 50 to about 1000 mg of AMP in one or two administrations, or an equivalent dose of another purine, and also enabling administration of an adequate dose of NSAID, e.g., a dose of about 50 to about 500 mg per day of aspirin, in one or two administrations, or an equivalent dose of another NSAID.

As an example, it is possible in an adult to administer a daily dose of about 50 to about 1000 mg of AMP and about 50 to about 500 mg of aspirin for a treatment that should last from about 2 to about 4 weeks. In the case of ad hoc use, it is possible to administer, e.g., via the oral or sublingual route, from about 200 to about 1000 mg of AMP and from about 100 to about 300 mg of aspirin in a single administration about 30 minutes to about 2 hours prior to the envisaged sexual relation.

AMP can be replaced notably by equivalent quantities of ATP.

If it is desired to substitute another purine for the AMP and/or another NSAID for aspirin, it is very easy to adjust the dose ranges mentioned above by replacing a given dose of AMP by an equivalent dose of another purine and/or replacing a given dose of aspirin by an equivalent dose of another NSAID. A purine dose equivalent to a given dose of AMP is, e.g., a purine dose capable or inducing a relaxation of the smooth muscles of the isolated rabbit corpus cavernosum (previously contracted with phenylephrine) in an organ chamber, this relaxation being comparable to that obtained with said given dose of AMP in a test using the known techniques described notably by HOLMQUIST et al., J. Urol. 144, 146-151 (1990); BRODERICK et al., Neuro. Urol. Urodyn. 10, 507-515 (1991); BUSH et al., 147, 1650-1655 (1992); HSI Yang Yu, Int. J. Impot. Res. 5, 161-167 (1993); SAENZ DE TEJADA et al., J. Pharmacol. Exp. Treat. 290(1), 1-8 (1999). An NSAID dose equivalent to a given dose of aspirin is, e.g., a dose which, in combination with a purine, is capable of inducing relaxation of the smooth muscles of the corpus cavernosum which is comparable to the relaxation obtained with the dose of aspirin combined with the same purine in a test using one of the techniques mentioned above.

The invention also pertains to a method for the prevention or treatment of male or female sexual dysfunctions in which a drug as defined above is administered in a therapeutically effective amount.

The invention also pertains to a nontherapeutic method for increasing sexual desire and/or sexual capacities and/or promoting sexual activity and/or improving the intensity of sexual pleasure and/or promoting the attainment of satisfying sexual relations in persons who so desire even though they do not suffer from sexual dysfunctions as defined above. This method comprises the act of administering to such persons a purine and an NSAID (or a composition combining a purine activity and an NSAID activity), notably AMP and aspirin, e.g., between about 30 minutes and about 2 hours prior to an envisaged sexual activity. The doses administered can be selected in the dose ranges specified above.

The following example illustrates the invention.

### EXAMPLE

Sachet of Powder for Drinkable Suspensions

Sachets of powder are prepared which contain:

| | |
|---|---|
| -AMP: | 400 mg |
| -Aspirin: | 250 mg |
| -Aromatized excipient: | 500 mg |

The AMP can be replaced by an equivalent quantity of ATP; the aspirin can be replaced by an equivalent dose of mefenamic acid, salicylic acid, diclofenac, ibuprofen, naproxen, sulindac or indomethacin.

It can be employed such that the content of one sachet is ingested daily after suspending in water. It is also possible to ingest the content of a supplementary sachet about 30 minutes to about 2 hours prior to an envisaged sexual activity.

PHARMACOLOGICAL STUDY

This study was performed in vitro on corpus cavernosum smooth muscles from male and female rabbits. The technique is that of organ chambers.

The objective of this study was the determination of the effect of purine (AMP and ATP) on the relaxation of the corpus cavernosum after a phenylephrine precontraction and the determined of a possible modification of this effect of the purines by aspirin. The study was performed on male and female rabbits.

In the experimental test, AMP induced a noteworthy relaxation of the corpus cavernosum sooth muscle of male rabbits which reached 70% at 10⁻³M. Aspirin at 10⁻³M potentiated this effect of AMP because the combination advanced the relaxation to 92%. This potentiation effect can not be explained by the addition of the response to AMP and the response to aspirin since aspirin itself is not vasoactive on this model.

Aspirin also potentiated the effect of ATP on the same test on male rabbits but the effect of ATP was found to be weaker than that of AMP (35% relaxation with ATP 10⁻³M, reaching 57% in the presence of aspirin 10⁻³M).

With regard to female rabbits, AMP (10⁻³M) induced a relaxation of 20%, reaching 36% in the presence of aspirin. ATP (10⁻³M) seemed to cause a somewhat greater relaxation of 31%, reaching 50% in the presence of aspirin.

In all cases, aspirin amplified the relaxant response of the purines studied (AMP or ATP). This amplification was of a factor ranging from 1.5 to 2.

Similar potentiating effects were observed when aspirin was replaced by mefenamic acid, salicylic acid or indomethacin.

1. A composition comprising a purine, an NSAID, and a pharmaceutical excipient and/or vehicle.

2. The composition according to claim 1, wherein the purine is selected from the group consisting of adenine, adenosine, guanine, guanosine, AMP, ADP, ATP, GMP, GDP and GTP.

3. The composition according to claim 1, wherein the NSAID is selected from the group consisting of salicylic acid derivatives, pyrazole derivatives, anthranilic acid derivatives, propionic acid derivatives, phenothiazine derivatives, indole derivatives, bucloxic acid, diclofenac and piroxicam.

4. The composition according to claim 1, wherein the composition is in the form of a capsule, drinkable solution, emulsion, granules, gel, cream, powder, tablet, compressed tablet, unguent, transdermal device, gynecological pessary, gynecological suppository, gynecological solution, injectable solution and injectable suspension.

5. The composition according to claim 1, wherein purine is selected from the group consisting of adenosine, AMP, ADP and ATP.

6. The composition according to claim 1, wherein purine is selected from the group consisting of AMP and ATP.

7. The composition according to claim 1, wherein the purine and the NSAID are linked according to formula (I)
(A—)ₘ(X)ₚ(—B)ₙ (I)
wherein A is a residue of an NSAID molecule, B is a residue of a purine and X represents either a covalent bond between A and B, or a spacer arm linking at least one A residue with at least one B residue, m is a whole number ranging from 1 to 3, n is a whole number ranging from 1 to 3, and p represents zero or a whole number equal at most to the larger of the numbers m and n.

8. The composition according to claim 1, comprising a dose of about 50 to about 1000 mg of the purine.

9. The composition according to claim 1, comprising a dose of about 50 to about 500 mg of the NSAID.

10. A method of treating male or female sexual dysfunction comprising administering a therapeutically effective amount of the composition according to claim 1 to a person.

11. The method according to claim 10, wherein the purine is selected from the group consisting of adenine, adenosine, guanine, guanosine, AMP, ADP, ATP, GMP, GDP and GTP.

12. The method according to claim 10, wherein the NSAID is selected from the group consisting of salicylic acid derivatives, pyrazole derivatives, anthranilic acid derivatives, propionic acid derivatives, phenothiazine derivatives, indole derivatives, bucloxic acid, diclofenac and piroxicam.

13. The method according to claim 10, wherein the composition is in the form of a capsule, drinkable solution, emulsion, granules, gel, cream, powder, tablet, compressed tablet, unguent, transdermal device, gynecological pessary, gynecological suppository, gynecological solution, injectable solution and injectable suspension.

14. The method according to claim 10, wherein purine is selected from the group consisting of adenosine, AMP, ADP and ATP.

15. The method according to claim 10, wherein purine is selected from the group consisting of AMP and ATP.

16. The method according to claim 10, wherein the purine and the NSAID are linked according to formula (I)
(A—)ₘ(X)ₚ(—B)ₙ (I)
wherein A is a residue of an NSAID molecule, B is a residue of a purine and X represents either a covalent bond between A and B, or a spacer arm linking at least one A residue with at least one B residue, m is a whole number ranging from 1 to 3, n is a whole number ranging from 1 to 3, and p represents zero or a whole number equal at most to the larger of the numbers m and n.

17. The method according to claim 10, comprising a dose of about 50 to about 1000 mg of the purine.

18. The method according to claim 10, comprising a dose of about 50 to about 500 mg of the NSAID.

19. A method of preventing male or female sexual dysfunction comprising administering a therapeutically effective amount of the composition according to claim 1 to a person.

20. The method according to claim 19, wherein the purine is selected from the group consisting of adenine, adenosine, guanine, guanosine, AMP, ADP, ATP, GMP, GDP and GTP.

21. The method according to claim 19, wherein the NSAID is selected from the group consisting of salicylic acid derivatives, pyrazole derivatives, anthranilic acid derivatives, propionic acid derivatives, phenothiazine derivatives, indole derivatives, bucloxic acid, diclofenac and piroxicam.

22. The method according to claim 19, wherein the composition is in the form of a capsule, drinkable solution, emulsion, granules, gel, cream, powder, tablet, compressed tablet, unguent, transdermal device, gynecological pessary, gynecological suppository, gynecological solution, injectable solution and injectable suspension.

23. The method according to claim 19, wherein purine is selected from the group consisting of adenosine, AMP, ADP and ATP.

24. The method according to claim 19, wherein purine is selected from the group consisting of AMP and ATP.

25. The method according to claim 19, wherein the purine and the NSAID are linked according to formula (I)
(A—)ₘ(X)ₚ(—B)ₙ (I)
wherein A is a residue of an NSAID molecule, B is a residue of a purine and X represents either a covalent bond between A and B, or a spacer arm linking at least one A residue with at least one B residue, m is a whole number ranging from 1 to 3, n is a whole number ranging from 1 to 3, and p represents zero or a whole number equal at most to the larger of the numbers m and n.

26. The method according to claim 19, comprising a dose of about 50 to about 1000 mg of the purine.

27. The method according to claim 19, comprising a dose of about 50 to about 500 mg of the NSAID.

28. A method for increasing sexual desire and/or promoting sexual activity and/or increasing sexual capacities and/or promoting sexual activity and/or improving the intensity of sexual pleasure and/or promoting the attainment of satisfying sexual relations in persons not suffering from sexual dysfunctions comprising administering to a person a composition comprising a purine and an NSAID.

29. The method according to claim 28, wherein the purine is selected from the group consisting of adenine, adenosine, guanine, guanosine, AMP, ADP, ATP, GMP, GDP and GTP.

30. The method according to claim 28, wherein the NSAID is selected from the group consisting of salicylic acid derivatives, pyrazole derivatives, anthranilic acid derivatives, propionic acid derivatives, phenothiazine derivatives, indole derivatives, bucloxic acid, diclofenac and piroxicam.

31. The method according to claim 28, wherein the composition is in the form of a capsule, drinkable solution, emulsion, granules, gel, cream, powder, tablet, compressed tablet, unguent, transdermal device, gynecological pessary, gynecological suppository, gynecological solution, injectable solution and injectable suspension.

32. The method according to claim 28, wherein purine is selected from the group consisting of adenosine, AMP, ADP and ATP.

33. The method according to claim 28, wherein purine is selected from the group consisting of AMP and ATP.

34. The method according to claim 28, wherein the purine and the NSAID are linked according to formula (I)
(A—)ₘ(X)ₚ(—B)ₙ (I)
wherein A is a residue of an NSAID molecule, B is a residue of a purine and X represents either a covalent bond between A and B, or a spacer arm linking at least one A residue with at least one B residue, m is a whole number ranging from 1 to 3, n is a whole number ranging from 1 to 3, and p represents zero or a whole number equal at most to the larger of the numbers m and n.

35. The method according to claim 28, comprising a dose of about 50 to about 1000 mg of the purine.

36. The method according to claim 28, comprising a dose of about 50 to about 500 mg of the NSAID.
